(19) United States
(12) Patent Application Publication
(10) Pub. No.: US 2009/0157010 A1
(43) Pub. Date: Jun. 18, 2009

(54) Stick-Resistant Medication Administration System

(76) Inventor: Robin P. Martin, McAllen, TX (US)

Correspondence Address: Raymond R. Ferrera, 1221 McKinney Street, Suite 4400, Houston, TX, 77010, US

(21) Appl. No.: 12/389,054

(22) Filed: Feb. 19, 2009

Related U.S. Application Data
(63) Continuation of application No. 11/897,153, filed on Aug. 29, 2007, now abandoned.
(60) Provisional application No. 60/933,191, filed on Jun. 5, 2007.

(51) Int. Cl. A61M 5/32

(52) U.S. Cl. 604/192

(57) ABSTRACT

A medication administration system is provided whereby a user can administer medication via intravenous, intramuscular, or subcutaneous routes from a single device in a safe and controlled manner, and without the danger of a needle-stick injury. The system essentially consists of a syringe-like device that is a closed system having an aseptic fluid path. The device includes a syringe body having an integrally disposed needle assembly and a plunger; a hollow casing with a syringe flange holder disposed at one end; a hollow needle-guard that is moveable both coaxially and telescopically within the casing and over the syringe assembly; an end cap which joins with the casing and holds the syringe assembly in a fixed position; an elastic means to urge the needle guard towards its most forward position; a locking means to lock the device while at rest; and a coupling adapter which joins at one end of the needle guard, limits the forward travel of the needle, creates a closed and aseptic fluid path to an IV port, and includes a means to join to the IV port, thereby completing the closed system without ever exposing the needle.

### RELATED APPLICATION DATA

The present application is a continuation of U.S. Non-Provisional application Ser. No. 11/897,153 filed Aug. 29, 2007, still pending, which claims the benefit of prior U.S. Provisional Application No. 60/933,191, filed Jun. 5, 2007, still pending.

### FIELD OF THE INVENTION

The present invention relates generally to medication administration systems, and in a particular though non-limiting embodiment, to a syringe-like device admitting to medication administration via intravenous, intramuscular, and/or subcutaneous routes in a safe and controlled manner, and without the danger of a needle-stick injury.

### BACKGROUND OF INVENTION

As will be readily appreciated by those of skill in the pertinent arts, contaminated needle-stick injuries constitute a longstanding and very serious problem in the healthcare industry. There are currently more than 20 known serious and potentially fatal blood-borne diseases transferable via needle-stick injury, the most serious being HIV and Hepatitis. According to one recent worldwide confidential survey, the problem remains widespread despite healthcare industry efforts to prevent accidental needle-sticks, with as many as one in four nurses in the United States reporting that they had received at least one needle-stick injury in the past 12 months. In Sub-Sahara Africa, where the spread of HIV/AIDS is an epidemic, nearly two-thirds of nurses reported accidental needle sticks.

Needle-sticks do not generally occur out of ignorance or because nurses do not know how to safely give an injection, but rather because presently employed methods of injection utilize exposed needles that are inherently very sharp and dangerous. In short, anytime a needle or other sharp object is exposed to a healthcare worker or another person, there is a risk of accidental needle-stick injury, often times depending on entirely unforeseeable conditions and circumstances, such as a bump from a co-worker, temporary loss of control of the device, patient movement, recapping, and so on. Many of the unforeseen circumstances that create needle-stick injuries are difficult, if not impossible, to anticipate or control. For these reasons and others, a better solution is required.

Prior efforts to prevent needle sticks have proven inadequate primarily because the needlepoint remains exposed to the healthcare worker during use of the device. It is generally only after an injection is fully performed that currently known devices offer a safety mechanism to cover or remove the needle from exposure in some fashion. According to the best figures available at this time, such designs have achieved a maximum reduction of no more than one-half of all needle-stick injuries, and offer no solution for the remaining one-half of needle-sticks that occur between the time a syringe is opened and completion of the medication injection.

### SUMMARY OF THE INVENTION

A medication administration system is provided whereby a user can administer medication via intravenous, intramuscular, or subcutaneous routes from a single device in a safe and controlled manner, and without the danger of a needle-stick injury. The system essentially consists of a syringe-like device that is a closed system having an aseptic fluid path. The device includes a syringe body having an integrally disposed needle assembly and a plunger; a hollow casing with a syringe flange holder disposed at one end; a hollow needle-guard that is moveable both coaxially and telescopically within the casing and over the syringe assembly; an end cap which joins with the casing and holds the syringe assembly in a fixed position; an elastic means to urge the needle guard towards its most forward position; a locking means to lock the device while at rest; and a coupling adapter which joins at one end of the needle guard, limits the forward travel of the needle, creates a closed and aseptic fluid path to an IV port, and includes a means to join to the IV port, thereby completing the closed system without ever exposing the needle.

### BRIEF DESCRIPTION OF THE DRAWINGS

FIG. 1 is a side view of an uncapped needle-stick resistant medication administration system according to the invention, depicted in an assembled state.

FIG. 2 is an exploded side view of a needle-stick resistant medication administration system according to the invention.

FIG. 3 is a syringe assembly as is already known in the art.

FIGS. 4a-4d depict various portions of the claimed device as would be suitable for practice in accord with the invention.

FIGS. 5a-5b depict raised views of a body portion suitable for practice in accord with the invention.

FIGS. 6a-6b are side views of an annular IV port-coupling device according to the invention.

FIGS. 7a-7c are various views of a threaded needle guard equipped with notched regions, suitable for engagement with matching protruding teeth contained in the threads of an anti-reuse cap.

### DETAILED DESCRIPTION OF THE PREFERRED EMBODIMENT

An object of the present invention is to provide a system in which a user can safely dispense medication in 3 of the 4 primary routes of administration (the main exception being inhalation), namely, by means of intravenous, intramuscular, or subcutaneous injection, without ever exposing the user to a needlepoint. In short, since at no point in time during operation or storage is the needlepoint exposed to the external environment, there is also no time during which an accidental needle-stick injury can occur.

Another object of the invention is to provide a medication administration system that can be safely used across a range of medication routes, including subcutaneous, intravenous, and intramuscular routes, all from the same device or device system.

A further object of the invention is to provide both passive and automatic means of returning a medication administration device to a safe-locked position any time the device is not in active use, even if an accidental loss of control of the device occurs.

It is a further object of the invention to provide a medication administration system design that lends itself to easy, cost-effective production tooling, as well as inexpensive and reliable manufacturing and assembly processes.

A further object of the present invention is to provide a medication administration device that can be fully disabled after completion of use, so that the device cannot be reused, in order to prevent the spread of secondary infections associated with the reuse of used needles.

In one particular, though non-limiting, embodiment, the objects of the invention described above and others are accomplished by a device comprising a conventional hypodermic syringe and needle assembly equipped with a plunger, surrounded by one or more of the following associated components: a primary assembly of a casing; a needle guard; an elastic means to constantly urge the needle guard to the forward most or most extended position; a locking means that automatically engages in the needle guard's forward-most position; and an end cap to hold the assembly together. Further embodiments comprise a secondary coupling device assembly present to maintain a closed system with an IV line utilizing a luer-type port connection, and to prevent exposure of the needle when using the device for intravenous medication administration.

In one embodiment, the mentioned casing substantially surrounds the syringe and needle assembly, and is open at each end. A first open end functions to engage with the needle guard telescopically and coaxially between a fully extended resting position, a plurality of partially retracted positions, and a fully retracted position. A second end of the casing is made to accept the flange of a syringe assembly, and to engage with the end cap in a locking frictional manner so that the assembly is securely held together.

In a further embodiment, the needle guard engages with the casing in a telescopic and coaxial manner through one end, and further comprises a locking means so that the whole device rests in an automatically locked position whenever it is not in active use. The needle guard also provides a surface against which an elastic pressure means applies either forward or rearward pressure by either pressing or pulling so as to constantly urge the needle guard towards a forward-most position.

In another example embodiment, the needle guard has an opening formed at either end. At one end, there is an opening to allow injection of the needle directly into an IV port made from a semi-permeable material, or instead into the coupling device of an IV line equipped with a luer-type valve configuration, which is commonly used in the industry. Additionally, the needle guard has, disposed at the same open end, a threaded means for securely attaching to the coupling device if desired, thereby achieving a closed system without exposure of the needle once coupled.

At the other end of the needle guard, there is a second open end dimensioned to accept, pass through, and clear the surface of the syringe and needle assembly. The needle guard also carries a means for keeping the needle guard and casing in relatively stable coaxial and telescopic alignment with a minimum of rotational movement. If the device is used for either intramuscular or subcutaneous injection, the coupling device is unnecessary, and the primary assembly is used to perform the injection directly, thereby establishing a closed system in which the needle is never exposed at any time during use to the healthcare worker.

According to further embodiments, the end cap has an outer surface resembling the dimensions of the casing end that accepts the flange of a syringe and needle assembly. The end cap has a center opening that allows placement of the end cap over and past the end of the plunger contained in the syringe and needle assembly. The end cap has one surface that engages and holds the flange of the syringe assembly firmly against the flange-accepting end of the casing. When fully assembled, an opposite surface lies approximately flush with the back-most surfaces of the casing. The end cap also has one or more teeth that bend inwardly during assembly, and snap outwardly upon reaching a set of receiving grooves or holes in the flange-receiving end of the casing, which, once snapped in place, are not easily disassembled, but which securely hold the entire assembly together.

Disposed between the needle guard and the casing is an elastic tension-delivering means suitable for either pushing or pulling against the two parts in such a manner that the needle guard is always urged forward along the inside of the casing towards a forward-most (i.e., fully extended) position within the casing. Typically, the elastic means will comprise a spring or other similar tension-delivering object, though other means of advancing the needle guard forward will also suffice provided said means satisfies the basic function of constantly urging the needle guard toward the front-most, fully extended, position within the casing body.

For example, the elastic means could also be accomplished with an elastic rubber band or other similar device without departing from the scope of the invention.

In other embodiments, the secondary assembly is formed in such a manner that there are at least two threaded portions, one disposed at either end, capable of engaging and attaching to external devices equipped with similar matching threads. The aforementioned devices might commonly include an IV port or the like, so that the threaded end of the primary assembly can be threaded and affixed to the IV port.

Disposed between the two threaded ends is an elastic, semi-permeable compound disposed in a fixed location such as is commonly used in IV ports. On the distal side of the elastic, semi-permeable compound is a threaded end that engages the primary assembly via a mechanism that limits the forward travel of the needle after passing through the elastic, semi-permeable compound to a predetermined fixed point, and which also forms one or more vents through which medication may pass within the closed aseptic system towards an IV port (for example, through a conduit designed to engage the luer-type port of an IV line, in a secure and liquid-tight seal).

The above-described medication administration system achieves many important and unexpected beneficial results. For example, the system enables the user to draw up and administer medication in a manner that maintains a continuous fluid path within the syringe and needle assembly in a closed, aseptic manner that effectively guards both the medication and the patient from introduction of foreign matter or bacteria.

By contrast, traditional syringes (including even safety syringes), provide little if any safeguards against the needle and medication becoming contaminated with bacteria or other foreign matter, since the needles are commonly exposed during use. Since infected injection sites are a relatively common occurrence in healthcare settings, and can produce very serious, long lasting, and difficult to treat infections, the present invention is suitably disposed to immediately fill a long-standing need within the industry for sterility that can only be achieved in closed systems, and safety that can only be achieved in a system in which the healthcare worker is never exposed to a needle.

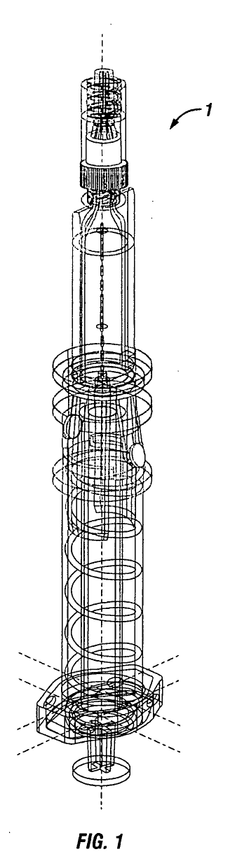

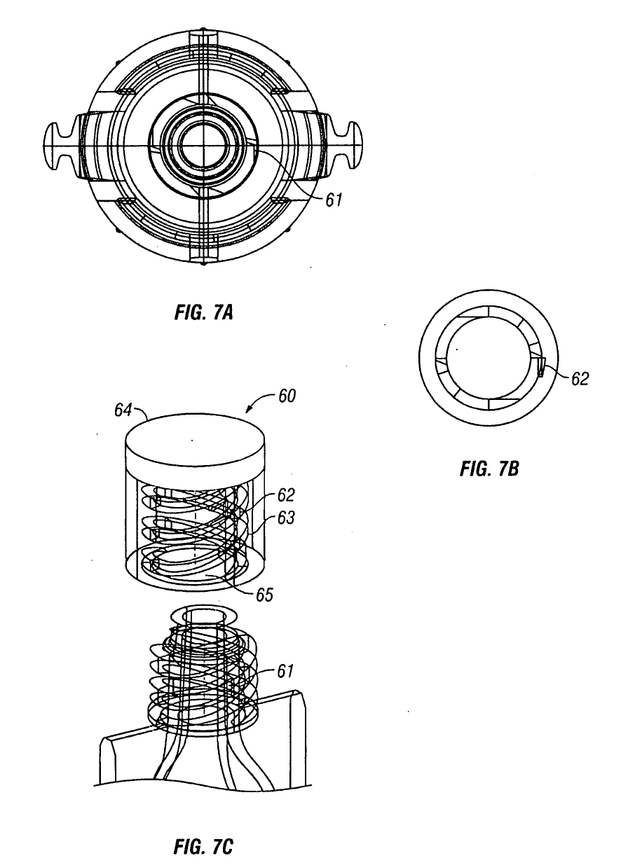

Referring now to the attached drawings for greater detail, the example embodiment in FIG. 1 depicts a complete needle-stick resistant medication administration system in an assembled state, with the exception of an anti-reuse cap, which is designated numeral 60 in FIG. 7c.

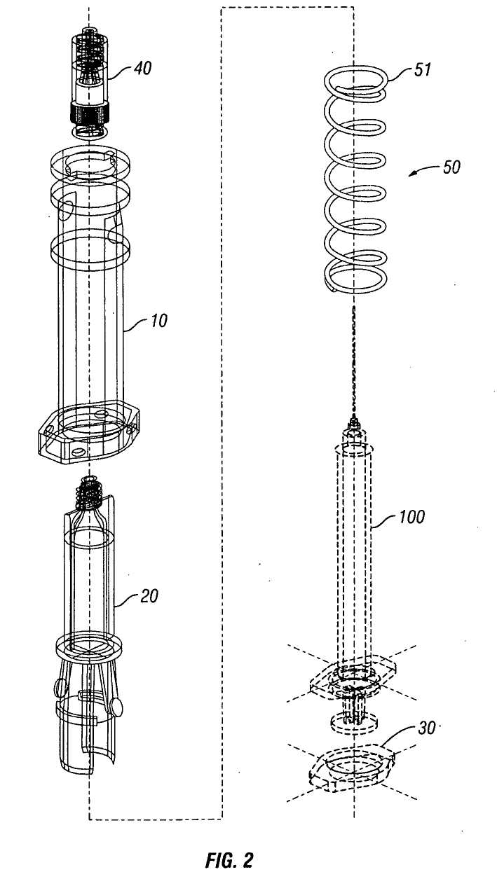

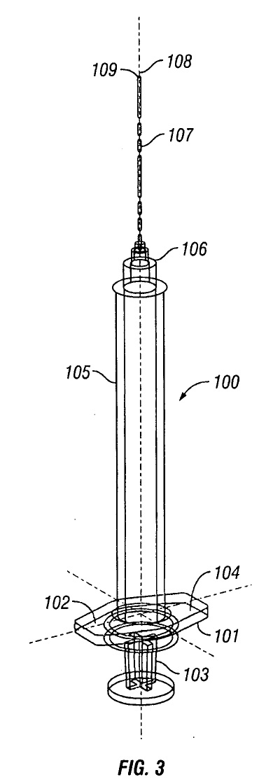

The example embodiment in FIG. 2 is an exploded side view of a needle-stick resistant medication administration system comprising a casing 10, a needle guard 20, an end cap 30, an IV port coupling device 40, and a spring 50. Depicted in FIG. 3 is closer view of known syringe 100, presented to show how the present invention can be designed around any conventional hypodermic syringe by simply adjusting the dimensions of the product, since there are many such syringes presently on the market.

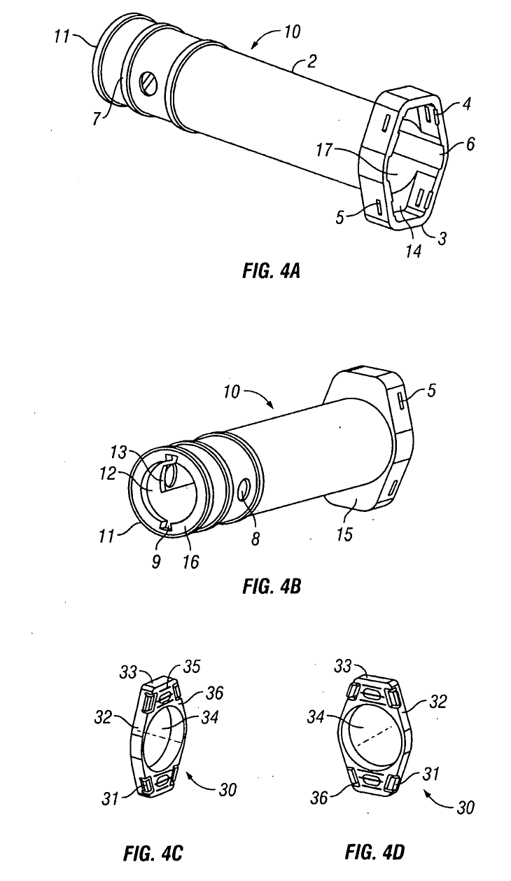

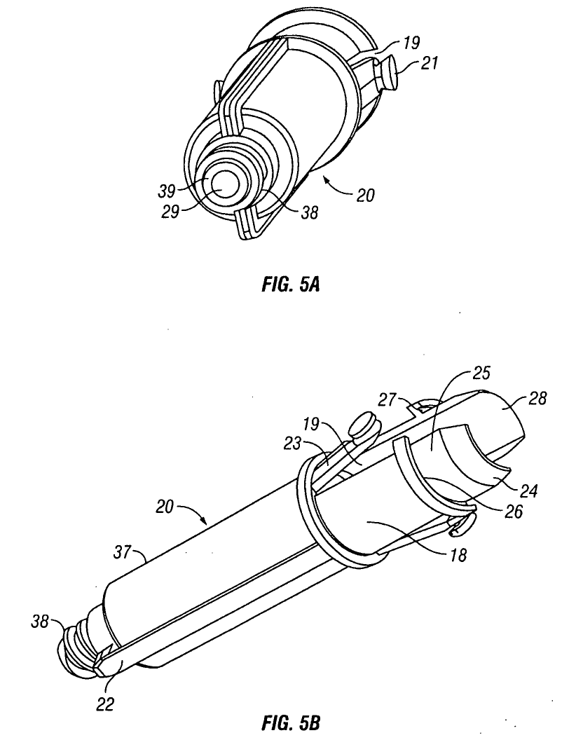

Referring now to the example embodiments depicted in FIGS. 4a-4d and FIGS. 5a-5b together, and starting with the casing assembly 10, a body portion 2 is formed in an elongated, approximately annular shape, with an irregularly-shaped end 3 designed to accept a syringe flange in such a manner as to align the syringe coaxially with the casing 10 and an associated needle-guard 20. In this particular embodiment, irregular end 3 further comprises one or more grooves designed to engage with one or more teeth 31 of end cap 30 in order to help center and locate end cap 30 just prior to assembly. As shown, irregular end 3 further comprises one or more holes 5 through which the teeth 31 of end cap 30 extend into and snap in place in a secure manner. Irregular end 3 also comprises one or more grooves 6 shaped to receive and align one or more locking tabs 21 disposed on needle guard 20.

In this particular embodiment, the casing 10 has one or more surfaces 14 formed on an inner portion of irregular end 3 disposed perpendicular to body portion 2, said surfaces 14 shaped to securely engage the forward flat surface of a syringe flange (see, for example, FIG. 3, item 102). Irregular end 3 also has one or more outer surfaces 15 disposed parallel to and capable of mating with surface 14, which are used by the operator to help hold the device in place. Irregular end 3 also has an opening or void space 17 defined by the inner outline of irregular end 3. Along the body 2 of casing 10, one or more rings 7 are formed, which are thicker areas along the body 2 to provide added stiffness, gripping surface, and to guard the locking tab(s) 21 from prolonged compression during packaging and shipment.

Formed within the length of body portion 2 are one or more holes 8, which pass through the body from an outside surface toward the inside surface. Hole(s) 8 are positioned to receive locking tab(s) 21 whenever needle-guard 20 is extended to a forward-most position, and which then prevent any unintentional back-out movement of the needle-guard 20. A second end 11 of casing 2 is approximately annular, and disposed in mechanical communication with a correspondingly shaped opening or void space 12. In this embodiment, opening 12 further comprises one or more grooves 9 shaped to receive one or more annular ribs 22 reciprocally located on needle-guard 20 in a coaxial, telescopic engagement that prevents rotation of needle-guard 20 with respect to casing 10, thereby maintaining coaxial alignment of locking tab(s) 21 with holes 8.

Near an end portion of groove 6 closest to hole 8, a sloped section 13 is shaped so as to engage a locking tab arm 23 at a corresponding angle. This feature helps to uniformly maintain the centering of needle-guard 20 between each inside surface of casing 10, which assists in the proper function and reliability of the device. Casing 10 comprises a top surface 16 disposed near round opening 12, which is angled slightly upward off the horizontal plain looking from the outer rim towards the inner rim. This feature aids in manufacturing by allowing the injection mold manufacturer to form a preferred tapered shut-off area in this locality, which greatly increases the reliability and longevity of the injection mold.

According to further embodiments, end cap 30 is formed having a left and right outer edge surface 32 that matches irregular end 3 of casing 10. End cap 30 also comprises a top and bottom outer edge surface 33 that matches the inner outline of irregular end 3 of casing 10. During assembly (and in particular, after a syringe 100 and spring 50 have first been inserted), irregular end 3 is faced upward, and then end cap 30 is put into position and located in irregular end 3, with teeth 31 facing downward toward irregular end 3. End cap 30 is then pressed into opening 3, and the teeth of end cap 30 bent inward until they reach the hole(s) 5 of irregular end 3, at which point the teeth 31 again snap outward due to memory of the material, and lock within hole(s) 5, and within opening 17, and cannot be again removed without substantial force. This latter aspect is the action that holds the full assembly together.

However, in most embodiments (for example, as the system is depicted in FIG. 4d with 4 such teeth 31 and four such holes 5), the device will still function even if only a single such tooth 31 and hole 5 are engaged. As shown, end cap 30 has a central opening 34, shaped to receive, pass through and allow operation of syringe plunger 103 (FIG. 3) after assembly is completed. During actual manufacturing, it has been found that syringe 100 usually has a varying thickness of syringe flange 102 that must be compensated for. Accordingly, end cap 30 also comprises one or more crush ribs 35, shaped to provide a portion that extends between the end cap inner surface 36 and the back surface 104 of syringe flange 102, and which will contact both surfaces, even when the thickness of syringe flange 102 varies slightly.

In further embodiments, crushed rib 35 is shaped in a manner that permits slight compression of any material disposed between inner surface 36 and syringe flange surface 104. This feature allows for each of the constituent assemblies to be firmly together in proper place and alignment, even with the variable thickness of syringe flange 102. This aspect of the system helps provide consistent and reliable functionality, with a low rejection rate of the devices during manufacturing, since free play of the syringe 100 within assembly 1 should generally be avoided. Around each tooth 31, there is an opening 36, shaped to match the outline of tooth 31, which allows free flexure of tooth 31 back and forth within the opening 36.

Referring now again briefly to FIGS. 1 and 2, once the device is assembled, a compression spring 50 is compressably disposed within the assembly between end cap 30 and the back end of needle-guard 20. Compression spring 50 supplies the constant forward urging force needed to push needle-guard 20 towards a fully extended and locked position. This arrangement achieves a fully automatic, though passive, safety feature of the system, whereby the device remains in a constantly state safety-locked position when not in use, or when disposed in an active though temporary rest position, or even during an instant in time when an operator may lose control of while performing an injection.

Referring now to FIGS. 5a-5b, it is seen that in this embodiment a needle-guard 20 is formed having a tapered rear arched surface 24 shaped to facilitate the assembly and centering of spring 50 in coaxial alignment with needle-guard 20 (and also with the aforementioned casing assembly 1). This embodiment also comprises a second arched surface 25, which matches closely the inside diameter contours of spring 50, upon which spring 50 rests during use, so that the spring compresses inwardly and outwardly as the device is used. Finally, needle-guard 20 has a slanted surface 26 against which a forward end 51 of spring 50 abuts, and thereby preventing additionally forward.

In one example embodiment, surface 26 contains a groove 27, shaped to receive a first coil end of spring end 51 in order to prevent misalignment of spring 50 within the assembly 1. In other embodiments, needle-guard 20 has a hollow passage between the rear and front of the part, beginning near a larger opening 28, and terminating near a smaller opening 29 formed near the front-most opening of the part.

In this embodiment, second arched surface 25 is joined with a third arched surface 18, which serves as a spacer in order to keep spring 50 pushed far enough back in the assembly so that the locking tab arm(s) 23 will freely flex both upwardly and downwardly within the assembly without interference of spring 50. An opening 19 surrounds locking tab arm 23, which also allows free flexure of locking tab arm 23 upwardly and downwardly without interference. Locking tab arm 23 generally comprises a memory material, such as a plastic, so that it will readily return back to its original position when flexed downward, away from its initial position, while in proximity of hole 8 in casing 10.

In a further embodiment, needle-guard 20 has a forward, elongated, hollowed section 37, shaped to accept the syringe barrel 105, syringe hub 106, and hollow bore needle assembly 107 within the hollow area. Preferably, hollow bore needle assembly 107 will pass through or past opening 29 whenever the device is not in use. The front end of needle-guard 20 is supplied with a threaded section 38 disposed near a front portion of the part, which is shaped to accept and engage matching threads 41 of an IV port coupling device 40 (see, for example, FIG. 7).

Near the forward-most end of needle-guard 20, a front end surface 39 is formed, which, due to the constant urging of needle-guard 20 toward a forward-most position, remains at all times in contact with the medication vial surface (or patient skin surface) while in use, thus preventing any exposure whatsoever of the dangerous and sharp needle-point 108 to the operator.

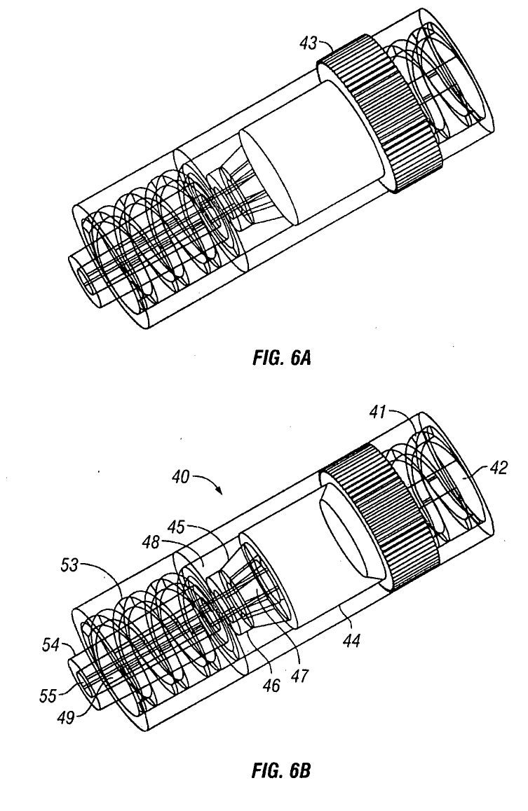

Moving on now to FIG. 6, an annular IV port-coupling device 40 comprises a rear portion 42 having an opening shaped to receive the threads 38 of needle-guard 20 within a set of threads 41. On the outside surface of the IV port-coupling device 40 is an outer portion 43 serrated around the outer surface in order to facilitate gripping and twisting of the device.

Near the inside end of threads 41 is an inner section 44 containing a semi-permeable thick elastic material that will permit insertion of needle-point 108 therethrough, while simultaneously creating a tight liquid seal around needle assembly 107. Near the forward end of section 44 is an opening 45, formed inside the assembly on an opposite side, in which the orifice 109 of hollow bore needle assembly 107 and needle-point 108 come to rest during use. Near the top of opening 45 is a top section 46 comprised of a solid material (for example, a rigid, inflexible plastic) that prevents any further forward travel of needle-point 107.

Disposed between the a beginning of opening 45 and solid material 46 is a connecting section, which also contains one or more vent openings 47, through which liquid contents of syringe 100 may pass within the assembly. Upon injection of the liquid contents of syringe 100 into the IV port coupling device 40, the liquid fills an inside chamber 48, and is then forced out of the device via opposite end 54 through a hollow tubular portion 49, which has been shaped to enter into and conformingly seal with a matching IV line port with a spring valve (not shown) through front orifice 55. The forward section of IV port coupling device 40 also contains forward threads 53, which are formed to engage the matching outside threads of a typical IV port and spring valve assembly, which is now commonly used in hospitals and elsewhere in the healthcare industry.

Referring now to FIGS. 7a-7c, a threaded portion 38 of needle guard 20 is equipped with one or more notched areas 61 that are notched slightly inward. These notched areas 61 are shaped to engage with matching protruding teeth areas 62 contained in the threads 63 of an anti-reuse cap 60. The anti-reuse cap 60 can be made of any suitable solid material, such as hard plastic. One end of anti reuse cap 60 has opening 65, shaped to receive the threads 38 of needle guard 20. The opposite end of anti reuse cap 60 is closed and made of a hard material, which will not permit puncture, by needlepoint 108.

Protruding teeth areas 62 are formed through the wall of the part, so that they flex outwardly during attachment of the anti-reuse cap 60 to the threaded portion 38 of needle guard 20. Once the protruding teeth 62 pass over notched areas 61 of threads 38, they cannot be reversed, as teeth 62 lock within notched areas 61 if an attempt is made to unscrew the anti-reuse cap 60.

In this manner, and through use of the anti-reuse cap at the end of the injection, the whole system becomes permanently sealed and disabled, and cannot be reused. The entire assembly can then be promptly disposed of in the nearest sharps container.

Because of the automatic and passive safety feature of the device, the system is foolproof and safely operable not only throughout the entirety of the injection procedure, but through all points in time both before and after use. The system will greatly curb (or even eliminate) needle stick injuries to housekeeping personnel or other workers, and will also eliminate injuries resulting from overfilled sharps containers. In short, accidental needle-stick injury of any kind is virtually eliminated with use of the present invention.

Furthermore, through the use of the various features and parts, the claimed system provides an essentially closed medication administration system that prevents the introduction of foreign material and most bacteria during the injection procedure, and also supplies a completely aseptic path of travel for the injection of the medication, regardless of which route of administration is used.

The foregoing specification is provided for illustrative purposes only, and is not intended to describe all possible aspects of the present invention. Moreover, while the invention has been shown and described in detail with respect to several exemplary embodiments, those of ordinary skill in the pertinent arts will appreciate that minor changes to the description, and various other modifications, omissions and additions may also be made without departing from either the spirit or scope thereof.

1. A stick-resistant medication administration system, said system comprising:
a hypodermic syringe and needle assembly, wherein said assembly is equipped with a plunger and further comprises at least one of the following associated components:
a primary casing assembly;
a needle guard;
an elastic means used to urge said needle guard forward within said primary casing assembly;
a locking means that engages when said needle guard is disposed in a forward-most position within said primary casing assembly; and
a securing means to hold said assembly together.

2. The stick-resistant medication administration system of claim 1, further comprising:
a secondary coupling device that achieves a closed system when coupled with an IV line.

3. The stick-resistant medication administration system of claim 2, wherein said secondary coupling device further comprises a threaded region.

4. The stick-resistant medication administration system of claim 2, wherein said secondary coupling device further comprises a luer-type port connection.

5. The stick-resistant medication administration system of claim 1, wherein said casing assembly further comprises a first open end that engages with said needle guard telescopically and coaxially, and admits to establishment of a fully extended resting position, a plurality of partially retracted positions, and a fully retracted position.

6. The stick-resistant medication administration system of claim 1, wherein said casing assembly further comprises a second end that accepts a flanged portion of a syringe assembly, and engages with an end cap in a locking manner.

7. The stick-resistant medication administration system of claim 1, wherein said needle guard further comprises a receiving surface against which an elastic tension means can be applied in order to urge said needle guard towards a forward-most position.

8. The stick-resistant medication administration system of claim 1, wherein said needle guard further comprises a threaded portion for securely attaching the system to an IV port.

9. The stick-resistant medication administration system of claim 1, wherein said elastic means further comprises a spring.

10. The stick-resistant medication administration system of claim 1, wherein said locking means further comprises a plurality of teeth for mating with a receiving means.

11. The stick-resistant medication administration system of claim 1, wherein said securing means further comprises an end cap.

12. A method of rendering a medication administration system stick-resistant, said method comprising:
disposing a hypodermic syringe and needle assembly in mechanical communication with a needle guard and a primary casing assembly equipped with a plunger;
equipping said needle guard with an elastic means used to urge said needle guard forward within said primary casing assembly, and a locking means that locks when said needle guard is disposed in a forward-most position within said primary casing assembly; and
securing said casing assembly together in a manner that substantially resists disassembly.

13. The method of rendering a medication administration system stick-resistant of claim 12, further comprising:
disposing a secondary coupling device in mechanical communication with said casing assembly in order to achieve a closed system when said medication administration system is coupled with an IV line.

14. The method of rendering a medication administration system stick-resistant of claim 13, further comprising:
equipping said secondary coupling device with a threaded region.

15. The method of rendering a medication administration system stick-resistant of claim 13, further comprising:
equipping said secondary coupling device with a luer-type port connection.

16. The method of rendering a medication administration system stick-resistant of claim 12, further comprising:
forming a first open end in said casing assembly; and
engaging said casing assembly in mechanical communication with said needle guard telescopically and coaxially, so that a fully extended resting position, a plurality of partially retracted positions, and a fully retracted position for the needle guard are established.

17. The method of rendering a medication administration system stick-resistant of claim 16, further comprising:
forming a second open end in said casing assembly that securely mates with a flanged portion of a syringe assembly, and engages with an end cap in a locking manner.

18. The method of rendering a medication administration system stick-resistant of claim 12, further comprising:
equipping said needle guard with a surface against which an elastic pressure means can be applied in order to urge said needle guard towards a forward-most position.

19. The method of rendering a medication administration system stick-resistant of claim 12, further comprising:
equipping said needle guard with a threaded portion for securely attaching the system to an IV port.

20. The method of rendering a medication administration system stick-resistant of claim 12, further comprising:
equipping said elastic means with a spring.

21. The method of rendering a medication administration system stick-resistant of claim 12, further comprising:
equipping said locking means with a plurality of teeth that securely mate with a receiving means.

22. The method of rendering a medication administration system stick-resistant of claim 12, further comprising:
securing said casing assembly together using an end cap.
